(19) United States
(12) Patent Application Publication
(10) Pub. No.: US 2026/0227365 A1
(43) Pub. Date: Aug. 6, 2026

(54) IMAGE DIAGNOSTIC APPARATUS, IMAGE DIAGNOSTIC METHOD, AND STORAGE MEDIUM

(71) Applicant: TERUMO KABUSHIKI KAISHA, Tokyo (JP)

(72) Inventor: Yasunori YAMASHITA, Fujinomiya Shizuoka (JP)

(21) Appl. No.: 19/575,799

(22) Filed: Mar. 23, 2026

Related U.S. Application Data
(63) Continuation of application No. PCT/JP2024/034298, filed on Sep. 26, 2024.

(30) Foreign Application Priority Data
Sep. 27, 2023 (JP) 2023-166435

(51) Int. Cl. G01N 29/06; G01N 29/24; G01N 29/46

(52) U.S. Cl. CPC G01N 29/0645; G01N 29/2475; G01N 29/46

(57) ABSTRACT

An image diagnosis apparatus includes a connection interface connectable to an image diagnosis catheter, a display, a memory storing a program and setting data indicating first and second display depths and first and second image settings, and a processor. The processor executes the program to receive a signal acquired from an ultrasound transmitter and receiver, convert a distribution of reflected waves in a radial direction indicated by the received signal into a brightness distribution, and generate a base tomographic image based on the brightness distribution. The processor generates first and second tomographic images of a luminal organ at the first and second display depths from the base tomographic image, executes image processing using the first and second image settings, and controls the display to display a screen showing the processed tomographic images along with information indicating a position of the luminal organ.

### CROSS-REFERENCE TO RELATED APPLICATION

This application is a continuation of International Patent Application No. PCT/JP2024/034298 filed September 26, 2024, which is based upon and claims the benefit of priority from Japanese Patent Application No. 2023-166435, filed September 27, 2023, the entire contents of which are incorporated herein by reference.

### TECHNICAL FIELD

The present disclosure relates to an image diagnosis apparatus, an image diagnosis method, and a storage medium.

### BACKGROUND ART

A medical catheter is used for diagnosis or treatment of a lesion area existing in a luminal organ such as a blood vessel or a vascular vessel. Ultrasound sensors or light receiving sensors are provided in medical catheters for diagnosis, and the catheters are moved into organs, and images based on signals obtained from the sensors are used for diagnosis.

Diagnostic imaging of blood vessels, in particular, among luminal organs, is indispensable for safely and reliably performing procedures such as Percutaneous Coronary Intervention (PCI). For this reason, intravascular imaging techniques such as intravascular ultrasound (IVUS) using medical catheters, optical coherence tomography (OCT), and the like are widely used in addition to angiography techniques for capturing images from outside the body using a contrast agent.

In recent years, cases using IVUS for diseases of not only arteries but also veins are increasing. There is a known technique of inserting an ultrasound imaging catheter into a vein and determining the acuteness of a thrombus in order to evaluate deep vein thrombosis.

There is also known to display, while displaying an ultrasound image obtained by inserting an ultrasound imaging catheter into a vein, an indicator for specifying anatomical features of a venous vessel and identifying the orientation of the ultrasound image.

There is a known system that when an ultrasound image obtained by inserting an ultrasound imaging catheter into a blood vessel is displayed, a user can select an image type in accordance with, for example, whether coronary artery structures or peripheral venous structures are to be observed. This system sets gain, contrast, and the like in accordance with the selected image type.

### SUMMARY

In a case where a vein is targeted, information regarding the size of the venous vessel diameter, the influence of pulsation, and another blood vessel outside the blood vessel (for example, an artery) is required, but it is not easy to present both the intravascular information and the extravascular information in one tomographic image with equivalent precision.

Embodiments of the present disclosure provide an image diagnosis apparatus, an image diagnosis method, and a storage medium capable of appropriately presenting information regarding the interior and exterior of an organ in diagnosis or treatment of a luminal organ using a medical catheter.

An image diagnosis apparatus includes: a connection interface connectable to an image diagnosis catheter for a luminal organ, the image diagnosis catheter including an ultrasound transmitter and receiver; a display; a memory that stores a program and setting data, the setting data indicating a first display depth, a first image setting corresponding to the first display depth, a second display depth different from the first display depth, and a second image setting corresponding to the second display depth; and a processor configured to execute the program to perform the steps of: receiving a signal acquired from the ultrasound transmitter and receiver via the connection interface, converting a distribution of reflected waves in a radial direction indicated by the received signal into a brightness distribution, and generating a base tomographic image of the luminal organ based on the brightness distribution, generating, from the base tomographic image, first and second tomographic images of the luminal organ at the first and second display depths, respectively, executing image processing on the first and second tomographic images using the first and second image settings, respectively, generating a screen showing the processed first and second tomographic images along with information indicating a position of the luminal organ corresponding to the processed first and second tomographic images, and controlling the display to display the generated screen.

According to the present disclosure, it is possible to appropriately present information regarding the interior and exterior of an organ in diagnosis or treatment of a luminal organ using a medical catheter.

### BRIEF DESCRIPTION OF DRAWINGS

FIG. 1 is a schematic diagram of an image diagnosis apparatus.

FIG. 2 is an explanatory diagram illustrating operation of a catheter.

FIG. 3 is a block diagram illustrating a configuration of an image processing apparatus.

FIG. 4 is a schematic diagram of a segmentation model.

FIG. 5 is a flowchart illustrating an example of a process executed by an image processing apparatus.

FIG. 6 is a flowchart illustrating an example of a process executed by an image processing apparatus.

FIG. 7 is a diagram illustrating an example of processing details for contrast adjustment on a tomographic image.

FIG. 8 is a diagram illustrating an example of processing details for gamma adjustment on a tomographic image.

FIG. 9 is a diagram illustrating an example of processing details for gain adjustment on a tomographic image.

FIG. 10 is a diagram illustrating an example of processing details for STC adjustment on a tomographic image.

FIG. 11 is a diagram illustrating an example of a screen displayed on a display apparatus.

FIG. 12 is a flowchart illustrating a process for receiving a setting of a display depth.

FIG. 13 is a diagram illustrating an example of a setting screen.

FIG. 14 is a flowchart illustrating an example of a process executed by an image processing apparatus according to a second embodiment.

FIG. 15 is a flowchart illustrating an example of a process executed by an image processing apparatus according to the second embodiment.

FIG. 16 is a diagram illustrating an example of a screen displayed on a display apparatus in the second embodiment.

FIG. 17 is a diagram illustrating an example of a screen displayed on a display apparatus in the second embodiment.

FIG. 18 is a flowchart illustrating an example of a process executed by an image processing apparatus according to a third embodiment.

FIG. 19 is a flowchart illustrating an example of a process executed by an image processing apparatus according to the third embodiment.

FIG. 20 is a flowchart illustrating an example of a process executed by an image processing apparatus according to the third embodiment.

FIG. 21 is a diagram illustrating an example of a screen displayed on a display apparatus in the third embodiment.

FIG. 22 is a diagram illustrating an example of a screen according to a modification example.

### DETAILED DESCRIPTION

Embodiments of an image diagnosis apparatus, a catheter image display method, and a computer program of the present disclosure will be described below with reference to the drawings. In the following embodiments, information processing for a blood vessel will be described as an example of a luminal organ, but needless to say, the luminal organ is not limited to the blood vessel.

### First Embodiment

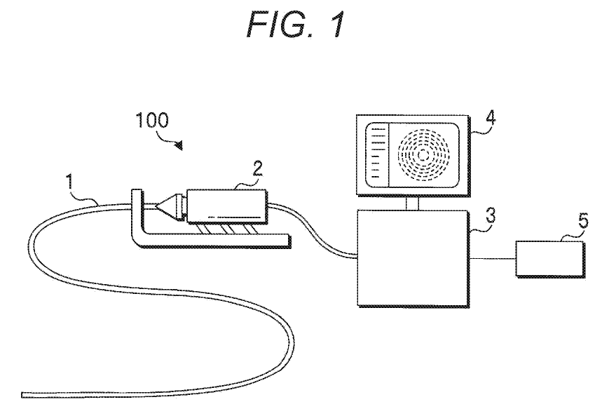

FIG. 1 is a schematic diagram of an image diagnosis apparatus 100 according to a first embodiment. The image diagnosis apparatus 100 includes a catheter 1, a motor drive unit (MDU) 2, an image processing apparatus 3, a display apparatus 4, and an input apparatus 5.

The catheter 1 is a medical flexible tube. The catheter 1 is referred to as an imaging catheter through which a shaft, having an imaging device 11 connected to a distal end thereof, is inserted. The imaging device 11 and the shaft in the catheter 1 are connected to the MDU 2 and the image processing apparatus 3 via a connector 12 on the proximal side.

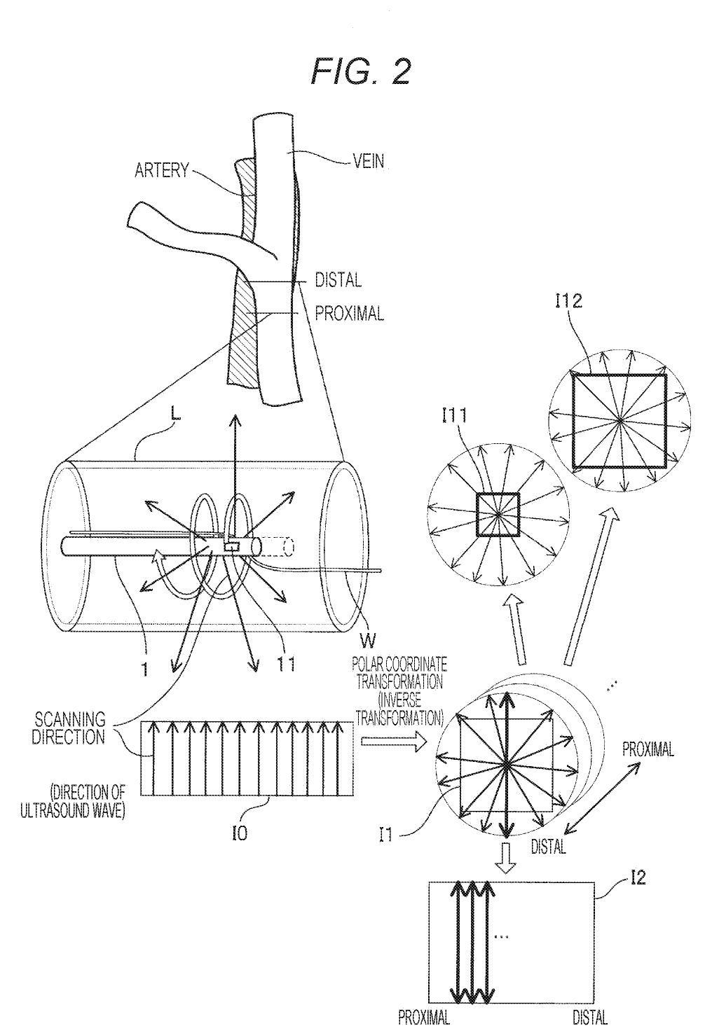

The imaging device 11 of the catheter 1 (see FIG. 2) includes an ultrasound probe including an ultrasound transducer and an ultrasound sensor of an IVUS method.

A signal obtained by the imaging device 11 of the catheter 1 is output to the proximal side of the catheter 1 via a signal line disposed in the shaft. The image processing apparatus 3 to which the catheter 1 is connected operates the imaging device 11 of the catheter 1, executes processing on the signal obtained from the imaging device 11, and displays an image generated by the processing on the display apparatus 4.

The MDU 2 is a drive apparatus attached to the proximal end of the catheter 1, and controls the operation of the catheter 1 by driving an internal motor in response to an operation by a physician or an examination operator.

The image processing apparatus 3 generates, from the signal obtained from the imaging device 11 of the catheter 1, an image obtained by converting a distribution of reflected waves in a radial direction from the inside of the luminal organ, into which the catheter 1 is inserted, into brightness, and performs polar coordinate transformation on the images generated for 360 degrees to generate a tomographic image (see FIG. 2). The image processing apparatus 3 outputs the generated tomographic image and information obtained by processing the tomographic image to a built-in display unit 35 or the externally connected display apparatus 4. The image processing apparatus 3 is, for example, a medical apparatus such as an intravascular image diagnosis apparatus, an angiography apparatus, an external monitor, or an electrocardiograph. The image processing apparatus 3 may be a smartphone, a tablet terminal, a laptop personal computer (PC), or a desktop PC, and functions as a medical apparatus such as an intravascular image diagnosis apparatus based on a software program in accordance with the application. Details of the configuration of the image processing apparatus 3 will be described later.

As the display apparatus 4, a liquid crystal display panel, an organic electro luminescence (EL) display panel, or the like is used. The display apparatus 4 displays a medical image generated by the image processing apparatus 3 and information regarding the medical image.

The input apparatus 5 is an input interface that receives an operation on the image processing apparatus 3. The input apparatus 5 may be a keyboard, a mouse, or the like, or may be a touch panel, a soft key, a hard key, or the like built into the display apparatus 4. The input apparatus 5 may receive an operation based on voice input. In this case, the input apparatus 5 uses a microphone and a speech recognition engine.

FIG. 2 is an explanatory diagram illustrating operation of the catheter 1. In FIG. 2, the catheter 1 is inserted into a tubular blood vessel L along a guide wire W inserted into the vein illustrated in FIG. 2 by a physician or an examination operator. The catheter 1 is moved within the blood vessel L as indicated by an arrow in the drawing by the drive of the MDU 2, and spirally scans the inside of the blood vessel with the imaging device 11.

In the image diagnosis apparatus 100 of the present embodiment, the image processing apparatus 3 acquires, for each scan, a signal output from the imaging device 11 of the catheter 1. One scan refers to emitting a detection wave from the imaging device 11 in a radial direction and detecting reflected light, and is performed spirally. The detection wave is emitted at an intensity that reaches the outside of the blood vessel L. The processing unit 30 of the image processing apparatus 3 performs logarithmic transformation on a waveform of the reflected wave, and obtains, for each scan, a brightness distribution in which an amplitude after the logarithmic transformation is converted into a brightness value.

The image processing apparatus 3 generates a tomographic image (cross-sectional image) (I1 in FIG. 2) obtained by performing polar coordinate transformation (inverse transformation) for every 360 degrees on a rectangular image (I0 in FIG. 2) in which brightness distributions for each scan are aligned in the radial direction for every 360 degrees and arranged in a rectangular shape. The tomographic image I1 is also referred to as a frame image. The reference point (center) of the tomographic image I1 corresponds to a range of the catheter 1 (which is not imaged). The image processing apparatus 3 may execute processing of generating the tomographic image I1 from the signal obtained from the imaging device 11 by specific hardware.

The image processing apparatus 3 may further generate a longitudinal image (longitudinal cross-sectional image) I2 in which pixel values on a straight line (an arrow indicated by a bold line) at an arbitrary angle passing through the reference point of the tomographic image I1 are arranged along a length direction (longitudinal axis direction) of the blood vessel by the catheter 1.

The image diagnosis apparatus 100 of the present disclosure is used for a physician to identify lesion areas and anatomical features inside and outside a vein, which is a luminal organ. Therefore, the examination operator or the physician visually recognizes the tomographic image I1 obtained by the imaging device 11 on the display apparatus 4 in real time while moving the catheter 1. Although it is difficult to display one tomographic image I1 while changing the display depth in real time, the image processing apparatus 3 displays tomographic images of different depths on the display apparatus 4.

In a case where a vein is an insertion target, it is important to observe a blood vessel itself of the vein illustrated in FIG. 2 and an organ existing outside the blood vessel, for example, an artery. Therefore, the image processing apparatus 3 further generates, from the generated tomographic image I1, a tomographic image I11 of a first display depth in which the inside of the blood vessel is a main observation target and a tomographic image I12 of a second display depth in which the outside of the blood vessel is a main observation target. The image processing apparatus 3 appropriately uses the obtained rectangular image I01, tomographic image I1, tomographic image I11, tomographic image I12, and longitudinal image, executes image processing, and outputs them in a manner that makes it easy to identify anatomical features and states of lesion areas in the vein. Hereinafter, the processing by the image processing apparatus 3 will be described in detail.

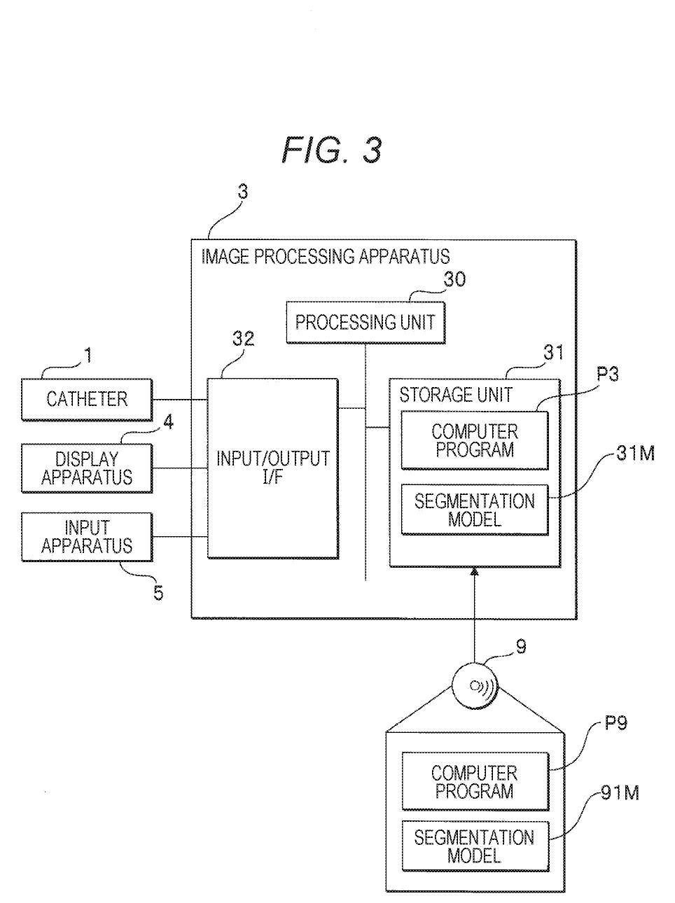

FIG. 3 is a block diagram illustrating a configuration of the image processing apparatus 3. The image processing apparatus 3 is a computer, and includes a processing unit 30, a storage unit 31, and an input/output I/F 32.

The processing unit 30 includes one or a plurality of central processing units (CPUs), micro-processing units (MPUs), graphics processing units (GPUs), general-purpose computing on graphics processing units (GPGPU), and tensor processing units (TPUs). The processing unit 30 incorporates a non-transitory storage medium such as a random access memory (RAM), and executes computation based on a computer program P3 stored in the storage unit 31 while storing data generated during processing in the non-transitory storage medium.

The storage unit 31 is a non-volatile storage medium such as a hard disk or a flash memory. The storage unit 31 stores a computer program P3 read by the processing unit 30, setting data, and the like. The setting data includes a first display depth and a second display depth. The first display depth is set to, for example, 10 mm, and the second display depth is set to, for example, 60 mm when a vein of a lower limb is targeted. The first display depth and the second display depth may be stored as a combination of a plurality of versions, and may be associated with data for identifying the combination. There may be a combination in which the first display depth is 2 mm and the second display depth is 60 mm, or a combination in which the first display depth is 5 mm and the second display depth is 60 mm. The setting data includes image settings of gain, contrast, Sensitivity Time Control (STC), and gamma correction for each of the first display depth and the second display depth. Furthermore, the storage unit 31 stores a trained segmentation model 31M. The segmentation model 31M will be described later.

The computer program P3 and the segmentation model 31M may be obtained by reading a computer program P9 and a segmentation model 91M stored in a non-transitory storage medium 9 outside the apparatus via the input/output I/F 32 and replicating the computer program P9 and the segmentation model 91M. The computer program P3 and the segmentation model 31M may be those distributed by a remote server apparatus and acquired by the image processing apparatus 3 via a communication unit (not illustrated) to be stored in the storage unit 31.

The input/output I/F 32 is an interface to which the catheter 1, the display apparatus 4, and the input apparatus 5 are connected. The processing unit 30 acquires signal data output from the imaging device 11 via the input/output I/F 32. The processing unit 30 outputs screen data of a screen including the generated tomographic images I1, I11, and I12, and/or the longitudinal image to the display apparatus 4 via the input/output I/F 32. The processing unit 30 receives operation information input to the input apparatus 5 via the input/output I/F 32.

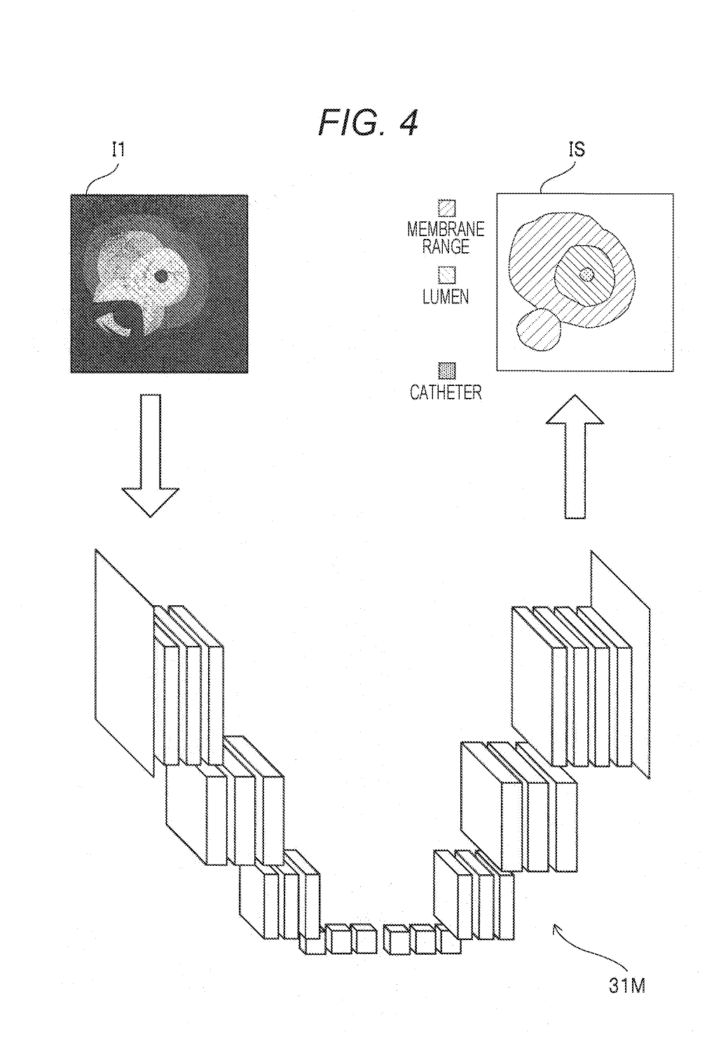

FIG. 4 is a schematic diagram of the segmentation model 31M. The segmentation model 31M is a model trained to output an image indicating a region of one or a plurality of target objects shown in an image when the tomographic image I1 (the tomographic images I11 and I12 may be used) is input. The segmentation model 31M is, for example, a model that performs semantic segmentation. The segmentation model 31M is designed to output, for each pixel in the input image, an image tagged with data indicating which target object each pixel belongs to.

As illustrated in FIG. 4, for example, a so-called U-net in which a convolution layer, a pooling layer, an upsampling layer, and a softmax layer are symmetrically arranged is used as the segmentation model 31M. The segmentation model 31M outputs a tag image IS1 in a case where the tomographic image I1 generated based on the signal from the catheter 1 is input. In the tag image IS1, the lumen range of the blood vessel, the membrane range corresponding to a space between the lumen boundary of the blood vessel including the media of the blood vessel and the blood vessel boundary, the range in which the guide wire W and its reverberation are present, and the range corresponding to the catheter 1 are tagged by assigning different pixel values (indicated by different types of hatching and blank in FIG. 4) to the pixels at their respective positions. The segmentation model 31M further identifies the range of a lipid-rich plaque formed in the blood vessel. The segmentation model 31M identifies a range in which a fibrous plaque or a calcified plaque appears.

Although the segmentation model 31M, the semantic segmentation and the U-net have been exemplified as described above, the segmentation model 31M is not limited thereto. In addition, the segmentation model 31M may be a model that implements individual recognition processing using instance segmentation or the like. The segmentation model 31M is not limited to the U-net base, and a model based on SegNet, R-CNN, an integrated model with other edge extraction processing, or the like may be used.

The processing unit 30 identifies the blood (lumen range), the intima range, and the adventitia range of the blood vessel appearing in the tomographic image I1 based on the pixel values in the tag image IS1 obtained by inputting the tomographic image I1 to the segmentation model 31M and the coordinates in the image. The processing unit 30 can detect the lumen boundary and the blood vessel boundary of the blood vessel appearing in the tomographic image I1 by identifying the range of the blood vessel. The blood vessel boundary is strictly the external elastic membrane (EEM) between the media and the adventitia of the blood vessel.

The processing unit 30 may identify each of the range of the lipid-rich plaque and the fibrous plaque or the calcified plaque based on the pixel values in the tag image IS1 obtained by inputting the tomographic image I1 to the segmentation model 31M and the coordinates in the image.

The processing unit 30 of the image processing apparatus 3 specifies the lumen boundary of the lumen range of the blood vessel from each range identified with respect to the tomographic images I1, I11, and I12, and calculates numerical values such as the maximum diameter, the minimum diameter, and the average inner diameter inside the lumen boundary. Moreover, the processing unit 30 can calculate, from the identification results of the ranges of the calcified plaque, the fibrous plaque, and the lipid-rich plaque identified for each of the IVUS tomographic images I1, I11, and I12, a ratio of the cross-sectional area thereof to the area inside the blood vessel boundary (hereinafter, referred to as a “plaque burden”). Specifically, for the tomographic images I1, I11, and I12, the plaque burden is calculated by the expression “1 - (lumen area/blood vessel boundary area)." The image processing apparatus 3 of the present disclosure may graphically output the distribution of the average lumen diameter with respect to the position of the blood vessel in the longitudinal axis direction and the distribution of plaque burden.

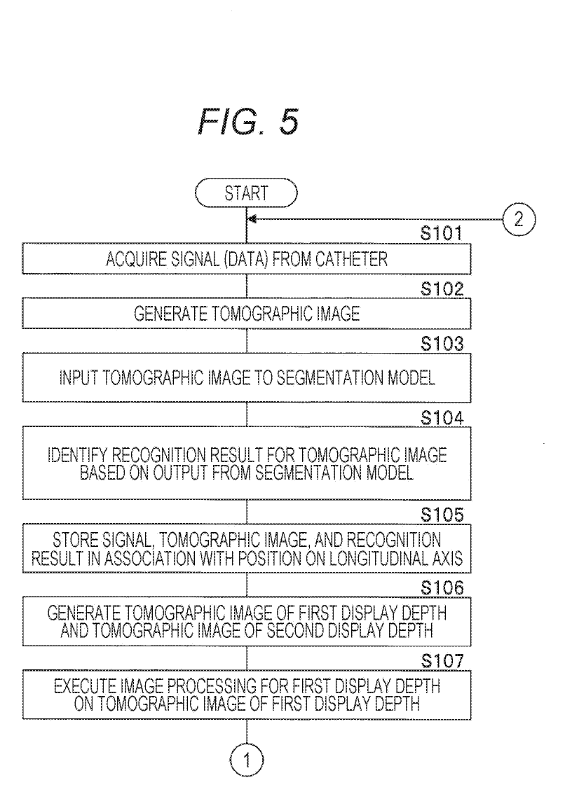

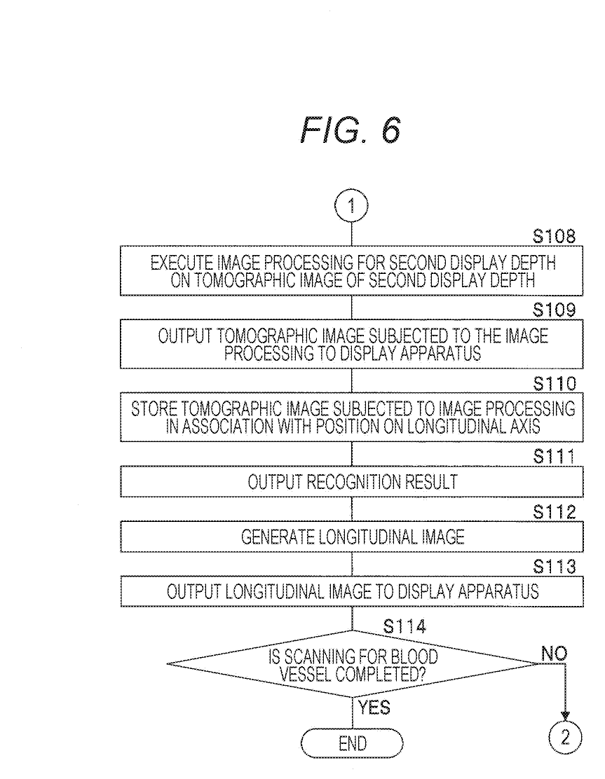

A process executed by the image processing apparatus 3 will be described with reference to a flowchart. FIGS. 5 and 6 are flowcharts illustrating an example of a process executed by the image processing apparatus 3. When an operation to start scanning of the image diagnosis apparatus 100 is performed and a signal is output from the imaging device 11 of the catheter 1, the processing unit 30 of the image processing apparatus 3 starts the following processing.

The processing unit 30 performs polar coordinate transformation (inverse transformation) on the rectangular images I0 arranged in a rectangular shape to generate the tomographic image I1 (S102) each time a predetermined amount (for example, 360 degrees) of signal data from the imaging device 11 of the catheter 1 is acquired (S101).

The processing unit 30 inputs the tomographic image I1 to the segmentation model 31M (S103). The processing unit 30 specifies the recognition result of the region appearing in the tomographic image I1 based on the tag image IS output from the segmentation model 31M (S104). In S104, the processing unit 30 specifies data indicating anatomical features such as the maximum value, the minimum value, and the average lumen diameter in the range inside the lumen boundary of the vein to be observed. In S104, the processing unit 30 may specify whether an artery appears outside the vein.

The processing unit 30 stores the signal data acquired in S101, the tomographic image I1, and the recognition result specified in S104 in the storage unit 31 in association with the data of the position on the longitudinal axis in the blood vessel (vein) (S105).

The processing unit 30 generates the tomographic image I11 of the first display depth and the tomographic image I12 of the second display depth, which are different in the radial direction, from the tomographic image I1 generated in S102 (S106). Since pieces of data indicating the first display depth and the data indicating the second display depth are included in the setting data of the storage unit 31 as described above, the processing unit 30 reads the pieces of data and generates the tomographic image I11 and the tomographic image I12 from the tomographic image I1.

The processing unit 30 executes image processing on the generated tomographic image I11 of the first display depth based on the image setting corresponding to the first display depth (S107). The image processing is at least one of gain adjustment, contrast adjustment, STC adjustment, that is, brightness adjustment, or gamma correction.

The processing unit 30 executes image processing on the tomographic image I12 of the second display depth based on the image setting corresponding to the second display depth (S108). Similarly, the image processing in S108 is at least one of gain adjustment, contrast adjustment, STC adjustment (that is, brightness adjustment), or gamma correction.

The processing unit 30 outputs the tomographic image I11 of the first display depth after the execution of the image processing in S107 and the tomographic image I12 of the second display depth after the execution of the image processing in S108 such that they can be displayed in real time on the screen displayed on the display apparatus 4 (S109).

The processing unit 30 stores the tomographic image I11 of the first display depth subjected to the image processing and the tomographic image I12 of the second display depth subjected to the image processing in the storage unit 31 in association with the data of the position on the longitudinal axis in the blood vessel (vein) (S110). Since the tomographic image I1 has already been stored in the storage unit 31, the processing of S110 is not essential.

The processing unit 30 outputs the text indicating the recognition result specified in S104 in accordance with the screen displayed on the display apparatus 4 in S109 (S111).

The processing unit 30 generates a longitudinal image I2 which is a longitudinal cross-sectional image at a predetermined angle based on the tomographic image I1 stored in association with the data of the position on the longitudinal axis of the blood vessel by the scanning performed so far (S112). In S112, the processing unit 30 may generate each of, or one of, the longitudinal images of the tomographic image I11 of the first display depth and the tomographic image I12 of the second display depth. The processing unit 30 outputs the generated longitudinal image I2 such that the generated longitudinal image I2 can be displayed in real time in a screen displayed on the display apparatus 4 (S113).

The processing unit 30 determines whether scanning by the imaging device 11 of the catheter 1 has been completed (S114). In a case where scanning is automatically performed in S114, the processing unit 30 determines whether the scanning has been performed for a set length. In S114, in a case where the examination operator or the physician performs an operation, the processing unit 30 determines whether the stop button has been pressed.

In a case where it is determined that the scanning is not completed (S114: NO), the processing unit 30 returns the processing to S101 and generates the next tomographic image I1. In a case where it is determined that the scanning is completed (S114: YES), the processing unit 30 ends the processing.

During the scanning or after the scanning is completed, the tomographic image I1 is stored in association with the position on the longitudinal axis of the blood vessel in S105. Therefore, the processing unit 30 may sequentially generate the longitudinal image I2 based on these images and output the longitudinal image I2 so as to be displayed on the display apparatus 4.

The processes illustrated in FIGS. 5 and 6 are examples, and the processing order may be partially changed as long as there is no inconsistency. Furthermore, in the processes illustrated in FIGS. 5 and 6, the processing unit 30 generates the tomographic image I11 of the first display depth and the tomographic image I12 of the second display depth (S106). However, in a case where S106 is omitted and the display at the first display depth is selected, while enlarging the range of the first display depth with respect to the tomographic image I1, the image processing for the first display depth may be performed and then the tomographic image I1 may be displayed on the display apparatus 4, and in a case where the display at the second display depth is selected, while enlarging the range of the second display depth with respect to the tomographic image I1, the image processing for the second display depth may be performed and then the tomographic image I1 may be displayed on the display apparatus 4. That is, the processing unit 30 may execute image processing in accordance with at which display depth the image is displayed on the display apparatus 4.

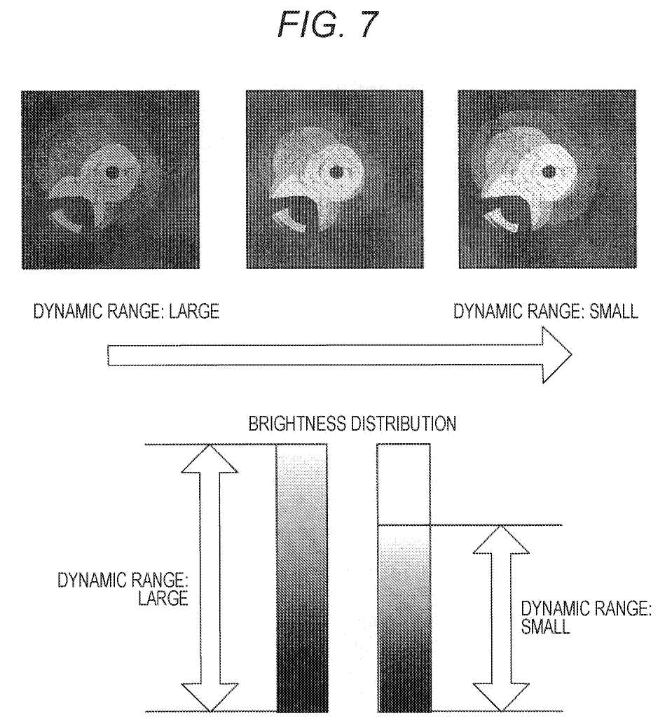

FIG. 7 is a diagram illustrating an example of processing details for contrast adjustment on the tomographic images I1, I11, and I12. The contrast is adjusted by changing the dynamic range for the brightness distribution. FIG. 7 illustrates a state where the contrast becomes stronger as the dynamic range is reduced. When the dynamic range with respect to the intensity of the ultrasound wave of the imaging device 11 is reduced, gradation of a soft tissue with a weak reflection intensity becomes clear, while a tissue harder than a certain level appears to have almost the same level of high brightness. Therefore, in the setting data, initially, the contrast is set to be adjusted to be strong with respect to the first display depth for a lesion area such as a plaque in the blood vessel, and the contrast is set to be weak with respect to the second display depth for detecting the presence or absence of another vessel wall outside the blood vessel.

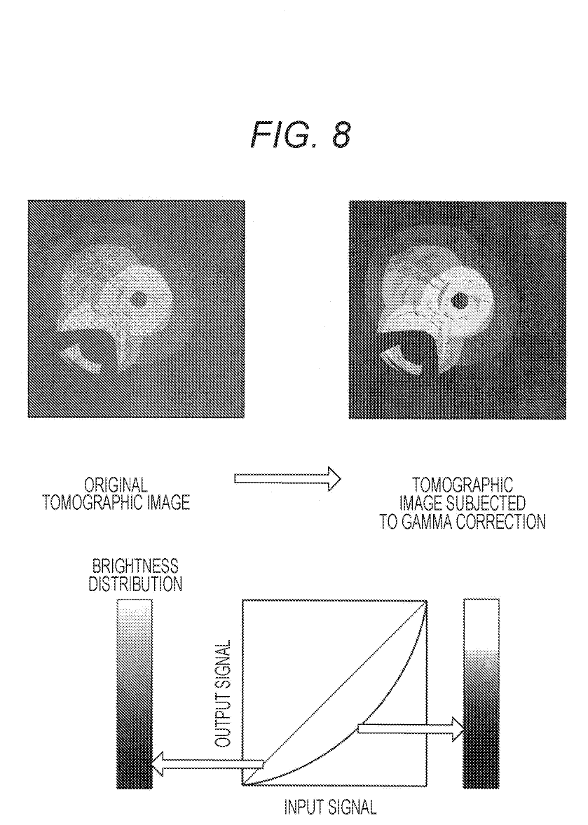

FIG. 8 is a diagram illustrating an example of processing details for gamma adjustment on the tomographic images I1, I11, and I12. The gamma adjustment is a correction for non-linearly converting the level of brightness, and by adjusting a gamma value, a conversion is performed to decrease a difference in a high brightness range and increase a difference in a low brightness range, or conversely, to decrease the difference in the low brightness range and increase the difference in the high brightness range. In the gamma adjustment illustrated in FIG. 8, the adjustment is executed to decrease a difference in a low brightness range and increase a difference in a high brightness range, from the original tomographic image on the left side to the corrected tomographic image on the right side. In this case, the conversion is performed such that a portion of pixel values in medium brightness becomes darker, and in the high brightness range, even a slight change leads to a large change in brightness to result in higher brightness. In the setting data, initially, the gamma value is set to be increased for the first display depth as illustrated in FIG. 8, and for the second display depth, the gamma value is set to a value smaller than that for the first display depth.

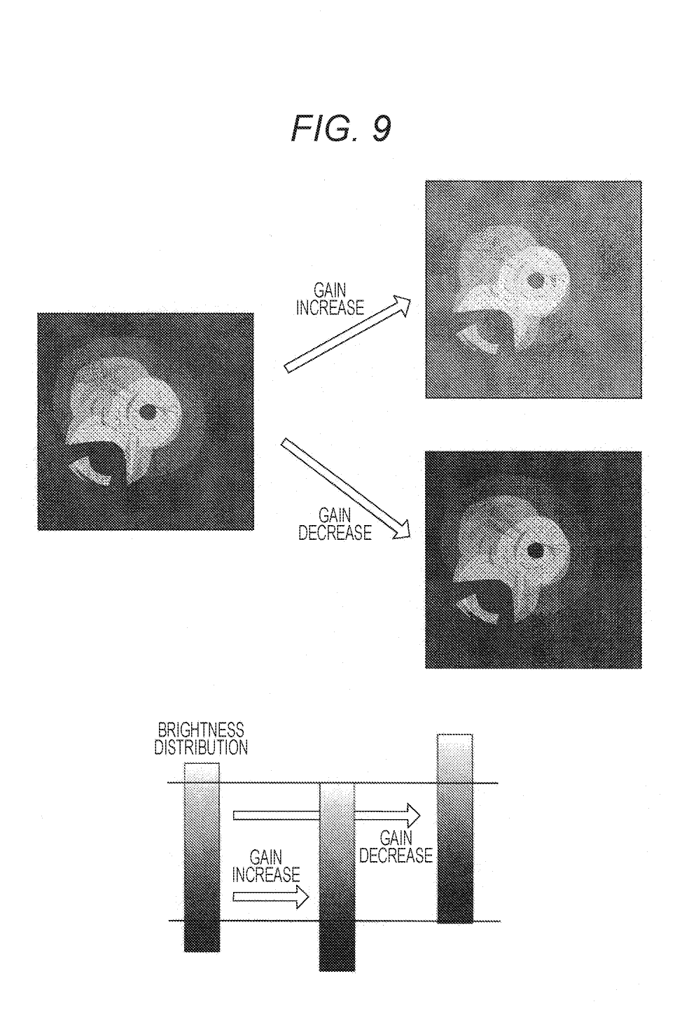

FIG. 9 is a diagram illustrating an example of processing details for gain adjustment on the tomographic images I1, I11, and I12. In the gain adjustment, an adjustment that amplifies the amplitude of a waveform of a reflected wave of an ultrasound wave to shift the level of brightness and the level of the amplitude upward or downward is performed. In the adjustment to increase the gain, the brightness value increases in a portion (low brightness) having a small amplitude of the reflected wave corresponding to a soft tissue, and the entire portion becomes bright. In the adjustment to decrease the gain, the brightness value decreases even when the amplitude of the reflected wave is medium (medium brightness), and the entire portion becomes dark. In the setting data, initially, the gain is set to be decreased for the first display depth, and the gain is set to be increased for the second display depth.

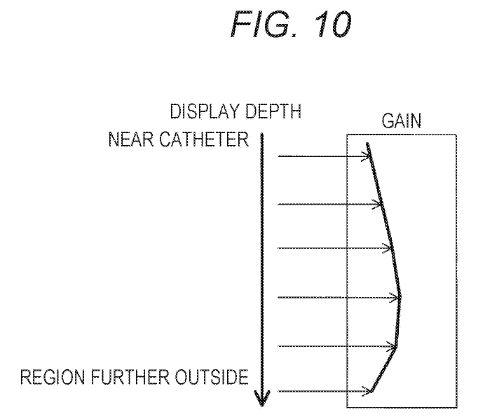

FIG. 10 is a diagram illustrating an example of processing details for STC adjustment on the tomographic images I1, I11, and I12. In the STC adjustment, the gain is changed for each display depth. In FIG. 10, the display depth is indicated by an arrow, and a range from the vicinity of the catheter 1 to the outside is indicated from an upper portion toward a lower portion on the arrow. In the STC adjustment illustrated in FIG. 10, the gain in the range from the middle portion to the outside is increased as compared with the vicinity of the catheter. For example, for the first display depth for observing the inside of the blood vessel, the membrane range becomes bright. In the setting data, initially, for the first display depth, the gain in the middle portion is increased to display the membrane range brightly as illustrated in FIG. 10, and for the second display depth with the outside of the blood vessel as a main observation target, the gain from the middle portion toward the outside is increased, and it is set such that whether another blood vessel (an artery) exists outside the blood vessel is easily recognized visually.

In the storage unit 31 of the image processing apparatus 3, the details of the various types of image processing illustrated in FIGS. 7 to 10 are stored while distinguishing between an image setting corresponding to the first display depth for mainly observing the inside of the blood vessel and an image setting corresponding to the second display depth for observing as far as the outside of the blood vessel.

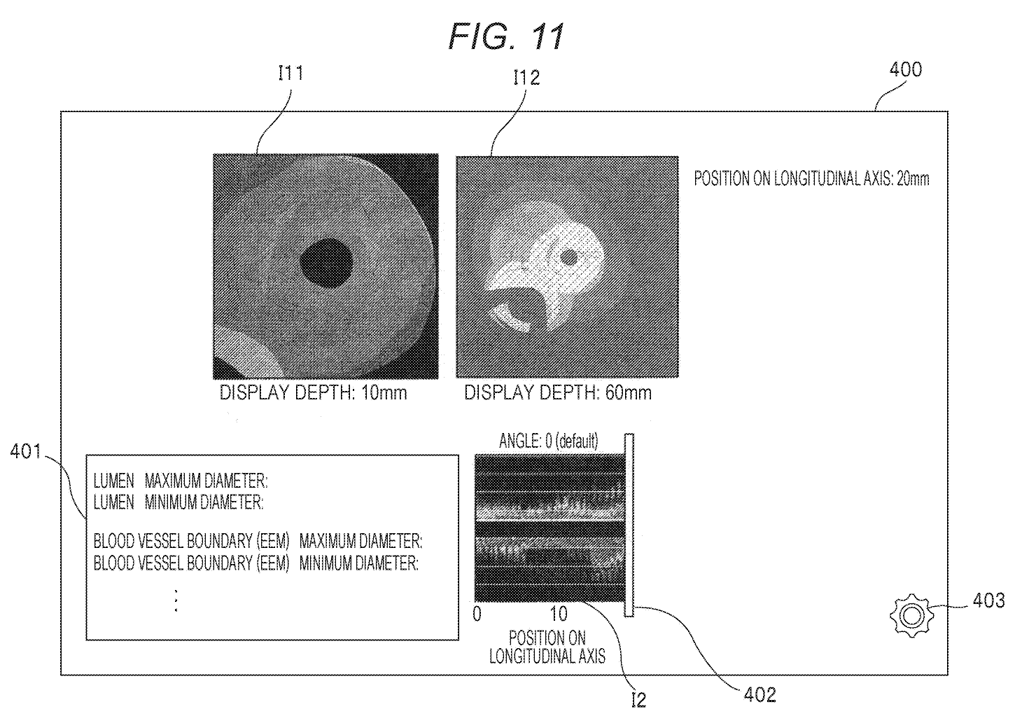

FIG. 11 is a diagram illustrating an example of a screen 400 displayed on the display apparatus 4. The screen 400 includes the tomographic images I11 and I12 of different display depths. In the example of FIG. 11, the display depth of the tomographic image I11 at the first display depth is 10 mm, and the display depth of the tomographic image I12 at the second display depth is 60 mm.

In the example of the screen 400 of FIG. 11, the tomographic image I11 of the first display depth for mainly observing the inside of the blood vessel is subjected to image processing of adjusting the gain while decreasing the brightness to sharpen the image. The tomographic image I12 of the second display depth for observing the outside of the blood vessel is subjected to image processing such that the brightness is increased to emphasize the brightness, and whether another blood vessel exists outside can be clearly seen.

The screen 400 further includes text 401 indicating data of the anatomical features specified based on the recognition results for the tomographic image I1. In the example of FIG. 11, the text indicates the maximum value and the minimum value of the lumen diameter at the position on the longitudinal axis at that time and the maximum value and the minimum value of the blood vessel boundary during real-time display.

The screen 400 further includes the longitudinal image I2 at a default angle up to a position on the longitudinal axis at that time. A cursor 402 indicating a position on the longitudinal axis at that time is superimposed and displayed on the longitudinal image I2.

The screen 400 includes a setting button 403. The setting button 403 may be selectable at any timing, or may be disabled so as to be unselectable during scanning of the blood vessel.

As illustrated in FIG. 11, in a case where the outside of the blood vessel is also to be observed, the tomographic image I11 of the first display depth for mainly observing the inside of the blood vessel and the tomographic image I12 of the second display depth for mainly observing the outside of the blood vessel are generated from the tomographic image I1, and both are displayed. Moreover, by performing appropriate image processing on both the inside and the outside of the blood vessel depending on the difference in display depth, it is possible to appropriately display information regarding the interior and the exterior of the blood vessel.

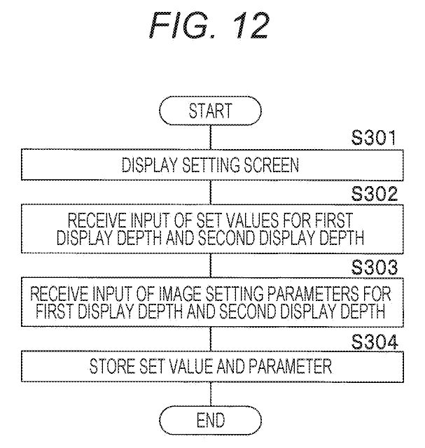

As described above, the first display depth and the second display depth are stored in the storage unit 31 as the setting data. The setting data can be changed by the examination operator or the physician before the start of scanning or during the operation. FIG. 12 is a flowchart illustrating an example of a processing procedure for receiving a setting of a display depth. In a case where the setting button is selected by the input apparatus 5, the processing unit 30 of the image processing apparatus 3 executes the following processing.

The processing unit 30 displays the setting screen (S301), and receives an input of a set value via the input apparatus 5 for the first display depth and the second display depth included in the setting screen (S302). The processing unit 30 receives an input of parameters of an image setting for each of the set first display depth and second display depth (S303). In S303, the processing unit 30 may receive an input of each of the gain, the contrast, the STC (brightness), and the gamma correction using a dial or a slide bar provided in the input apparatus 5.

The processing unit 30 stores the set values of the first display depth and the second display depth and the parameters related to the image setting in the storage unit 31 as setting data (S304), and ends the processing.

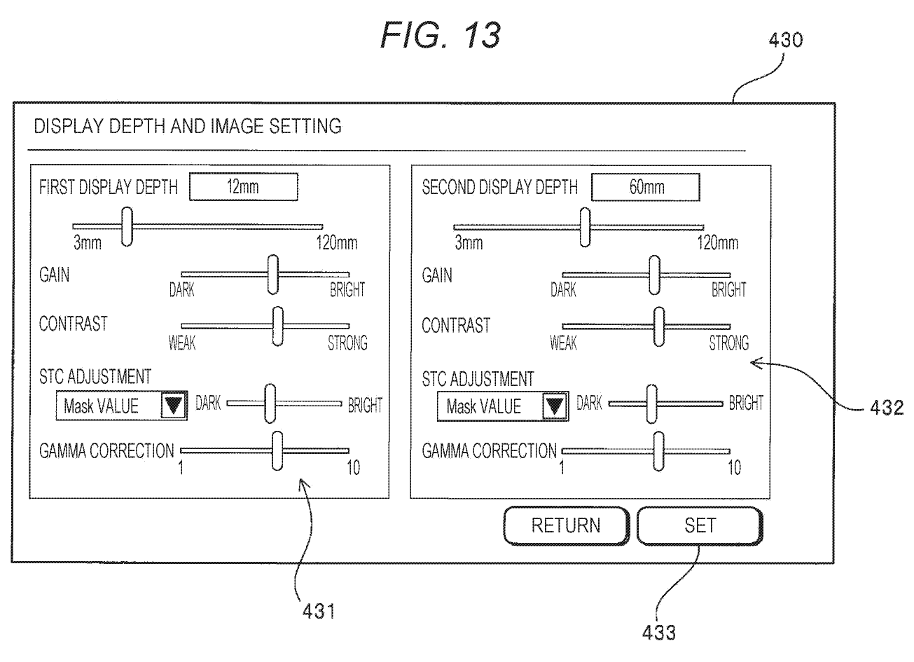

FIG. 13 is a diagram illustrating an example of a setting screen 430. As illustrated in FIG. 13, the setting screen 430 includes, for each of the first display depth and the second display depth, a first area 431 including a slide bar for adjusting a set value of the display depth, and a slide bar for adjusting gain, contrast, STC, and gamma correction, and a second area 432. In the adjustment of the STC, a parameter for gain adjustment (see FIG. 10) is received for each of a Mask value in a range corresponding to the catheter 1 and a depth based on the Mask value.

The setting screen 430 includes a setting button 433. When the setting button 433 is selected, the processing unit 30 stores the input set values and parameters.

By making the setting data including the first display depth and the second display depth adjustable, it is possible to display the blood vessel (vein) to be observed with a setting that is easy for the examination operator and the physician to visually recognize.

As described above, the image processing apparatus 3 provides a specific technological improvement in the field of medical imaging. Conventionally, it is difficult to present both intravascular information and extravascular information in a single tomographic image with equivalent precision, as the optimal image settings for observing the interior of a luminal organ differ from those for observing structures outside the luminal organ. The image processing apparatus 3 addresses it by generating a base tomographic image, generating first and second tomographic images at different display depths from the base tomographic image, and executing distinct image processing on each image using different image settings (such as gain, contrast, STC, and gamma correction). By simultaneously displaying these distinctly processed tomographic images on the screen, the apparatus improves the functioning of the imaging system itself and provides an enhanced graphical user interface that enables an operator to clearly and simultaneously visually recognize both the interior of the luminal organ and external anatomical features, such as another blood vessel.

### Second Embodiment

In a second embodiment, the image processing apparatus 3 does not constantly output the tomographic image I11 of the first display depth and the tomographic image I12 of the second display depth, and switches the images as necessary.

The configuration of the image diagnosis apparatus 100 according to the second embodiment is similar to that of the image diagnosis apparatus 100 according to the first embodiment except for the following processing procedure and display details. Therefore, in the image diagnosis apparatus 100 according to the second embodiment, the same reference numerals are given to the common components, and the detailed description thereof will be omitted.

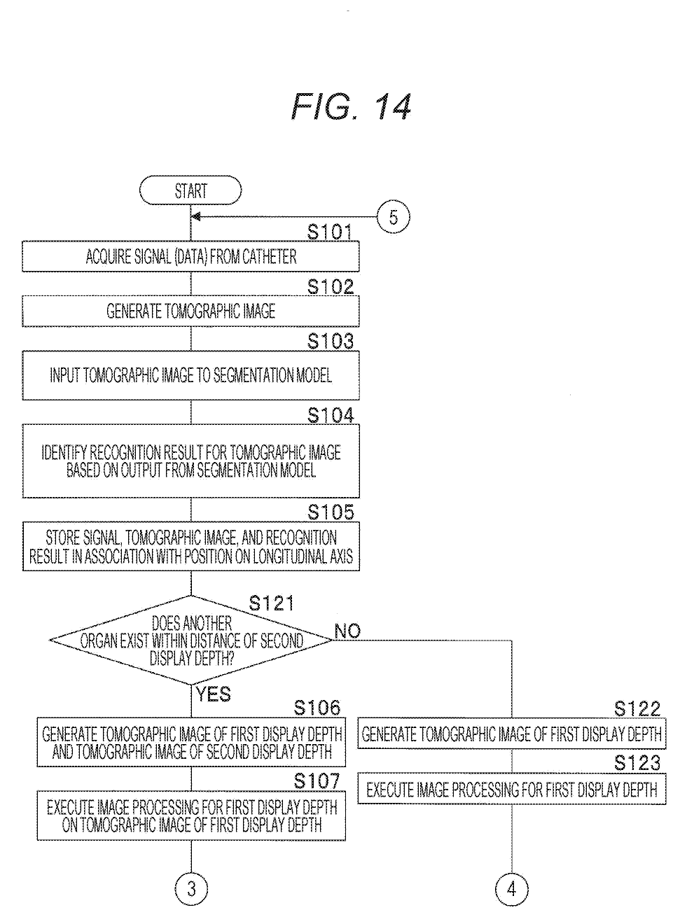

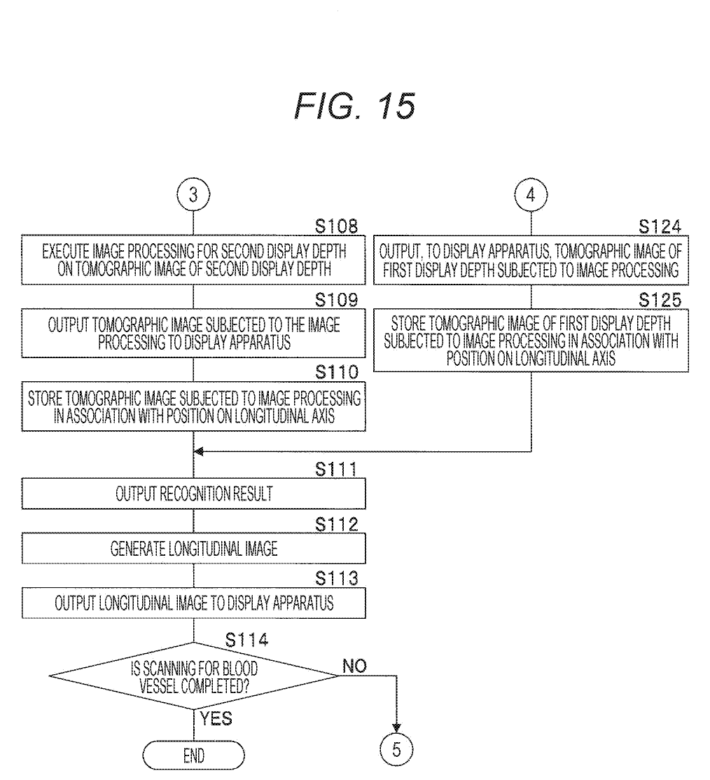

FIGS. 14 and 15 are flowcharts illustrating an example of the process executed by the image processing apparatus 3 according to the second embodiment. When an operation to start scanning by the image diagnosis apparatus 100 is performed via the input apparatus 5 and a signal is output from the imaging device 11 of the catheter 1, the processing unit 30 of the image processing apparatus 3 starts the following processing. Among the steps illustrated in the flowcharts of FIGS. 14 and 15, steps common to the process illustrated in the flowcharts of FIGS. 5 and 6 of the first embodiment are denoted by the same step numbers, and detailed description thereof will be omitted.

The processing unit 30 of the image processing apparatus 3 stores a signal data, the tomographic image I1 generated based on the signal data, and the recognition result based on the segmentation model 31M in the storage unit 31 (S105). Based on the stored data, the processing unit 30 determines whether another organ, specifically an artery, exists within a distance corresponding to the second display depth outside the organ into which the catheter 1 is inserted (S121). In S121, the processing unit 30 determines whether the region recognized as the artery is included in the tomographic image I1 generated from the reflection of the ultrasound waves up to the second display depth, thereby determining whether the artery exists.

In a case where it is determined that the other organ exists within the distance corresponding to the second display depth (S121: YES), the processing unit 30 generates the tomographic image I11 of the first display depth and the tomographic image I12 of the second display depth, which are different in the radial direction, from the tomographic image I1 (S106).

The processing unit 30 executes image processing on the generated tomographic image I11 of the first display depth based on the image setting corresponding to the first display depth (S107), and executes image processing on the tomographic image I12 of the second display depth based on the image setting corresponding to the second display depth (S108).

The processing unit 30 outputs the tomographic image I11 of the first display depth subjected to the image processing and the tomographic image I12 of the second display depth subjected to the image processing such that they can be displayed in real time on the screen displayed on the display apparatus 4 (S109). The processing unit 30 stores the tomographic image I11 of the first display depth subjected to the image processing and the tomographic image I12 of the second display depth subjected to the image processing (S110).

The processing unit 30 outputs text indicating the recognition result to the display apparatus 4 (S111). The processing unit 30 generates the longitudinal image I2 (S112), outputs the generated longitudinal image I2 to the display apparatus 4 (S113), and advances the processing to S114.

In S121, in a case where it is determined that there is no other organ within the distance corresponding to the second display depth (S121: NO), the processing unit 30 generates the tomographic image I11 of the first display depth for mainly observing the blood vessel from the tomographic image I1 (S122). The processing unit 30 executes image processing on the generated tomographic image I11 of the first display depth based on the image setting corresponding to the first display depth (S123).

The processing unit 30 outputs the tomographic image I11 of the first display depth after the execution of the image processing in S123 such that the image can be displayed in real time on the screen displayed on the display apparatus 4 (S124). The processing unit 30 stores the tomographic image of the first display depth subjected to the image processing in the storage unit 31 in association with the data of the position on the longitudinal axis in the blood vessel (S125). Since the tomographic image I1 has already been stored in the storage unit 31, the processing of S125 is not essential.

The processing unit 30 advances the processing to S111. Thus, only in a case where another organ appears in the range of the second display depth, the tomographic image I11 of the first display depth and the tomographic image I12 of the second display depth are displayed together on the display apparatus 4.

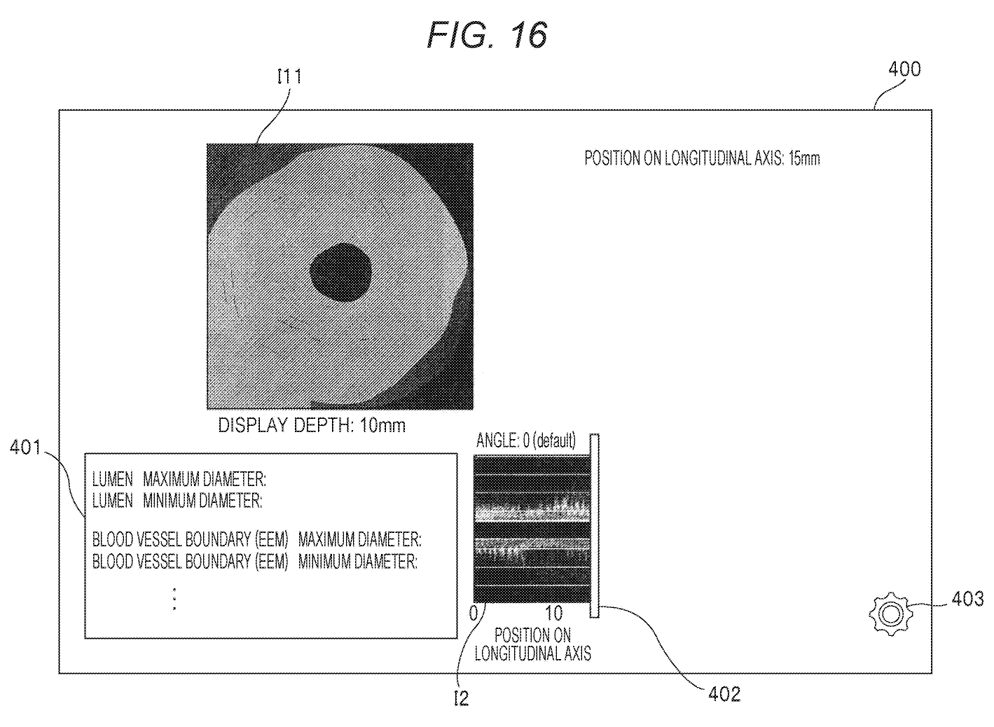

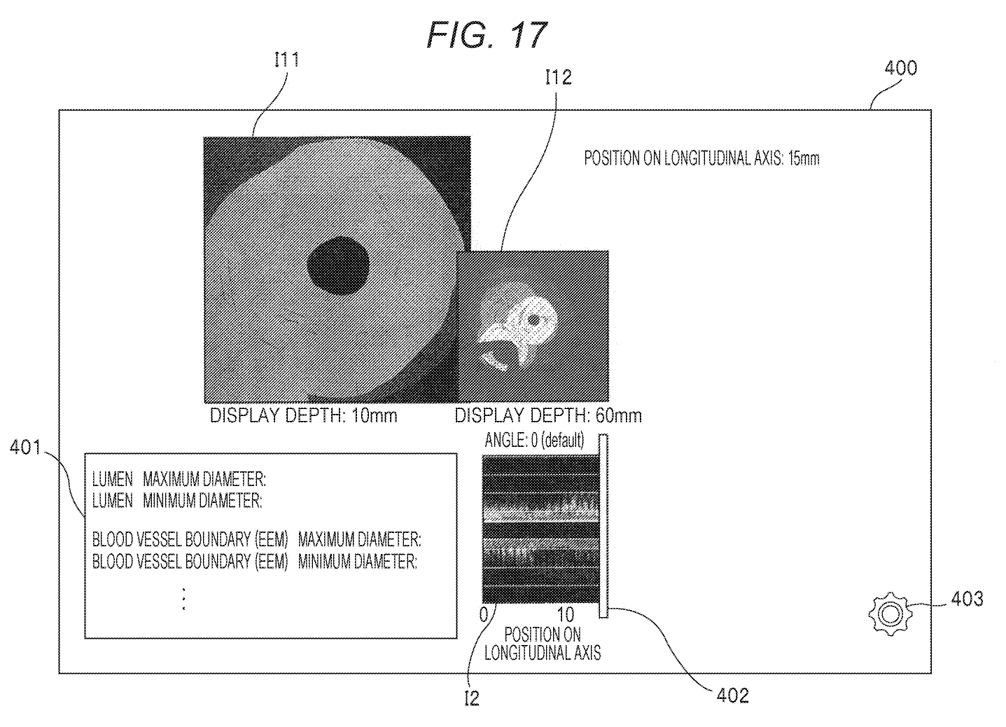

FIGS. 16 and 17 are diagrams illustrating an example of the screen 400 displayed on the display apparatus 4 in the second embodiment. The screen 400 of FIG. 16 and the screen 400 of FIG. 17 illustrate examples that change in response to the position of the catheter 1 in the blood vessel into which the catheter 1 is inserted or in response to the lapse of time. Similar to the example of the screen 400 illustrated in FIG. 11 of the first embodiment, the screen 400 illustrated in FIGS. 16 and 17 includes at least the tomographic image I11 and includes the text 401 indicating data of anatomical features, the longitudinal image I2, and the cursor 402.

FIG. 16 is a display example in a case where it is determined that there is no other blood vessel (artery) within the distance corresponding to the second display depth outside the blood vessel on the distal side of the blood vessel into which the catheter 1 is inserted. In FIG. 16, since it is determined that there is no other blood vessel outside the blood vessel, the tomographic image I11 of the first display depth is displayed in a large size, and is displayed with low brightness such that the contrast in the blood vessel becomes clear. Thus, it is possible to observe the state of the interior of the blood vessel and the blood cells.

FIG. 17 is a display example in a case where it is determined that another blood vessel (artery) exists. In FIG. 17, in addition to the tomographic image I11 of the first display depth illustrated in FIG. 16, the tomographic image I12 of the second display depth which allows observation of a wide range is displayed in parallel. When it is determined by the processing unit 30 that there is another blood vessel and the tomographic image I12 is generated, a notification may be displayed to notify the existence of the other blood vessel by showing the tomographic image I12 next to the tomographic image I11 along with a sound effect. The examination operator or the physician can continuously observe the tomographic image I11 while grasping the arrangement of the blood vessels and recognizing that there is a high possibility that a blood vessel exists outside without changing the setting manually.

### Third Embodiment

In a third embodiment, the display/non-display of each of the tomographic image I11 of the first display depth and the tomographic image I12 of the second display depth is switched in response to a selection operation.

The configuration of the image diagnosis apparatus 100 according to the third embodiment is similar to that of the image diagnosis apparatus 100 according to the first embodiment except for the following processing procedure and display details. Therefore, in the image diagnosis apparatus 100 according to the third embodiment, the same reference numerals are given to the common components, and the detailed description thereof will be omitted.

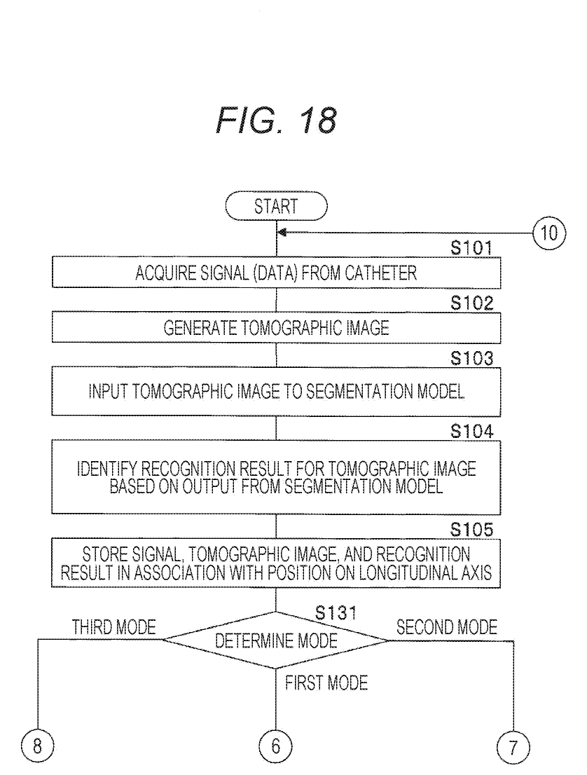

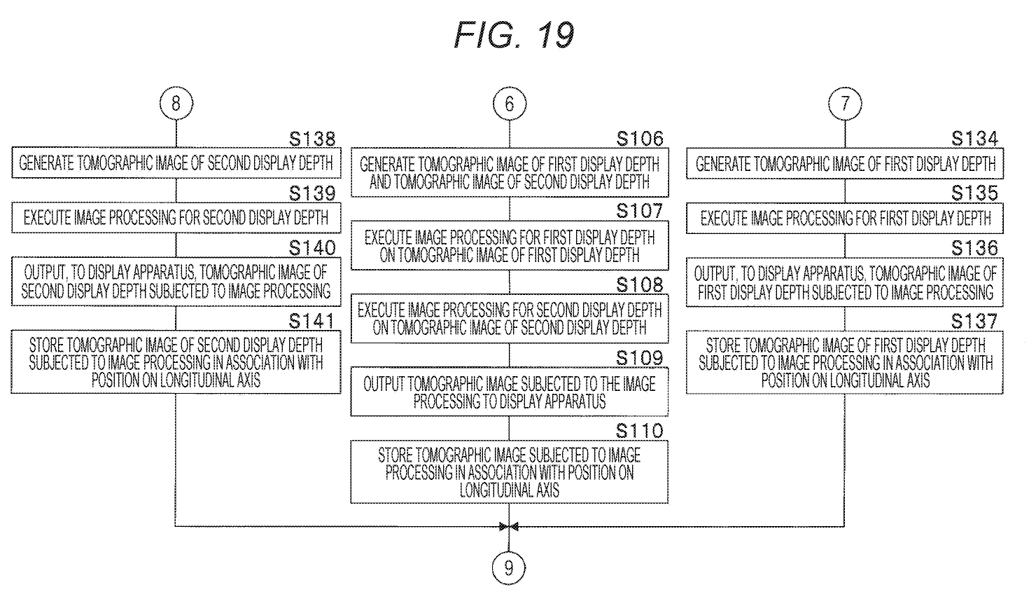

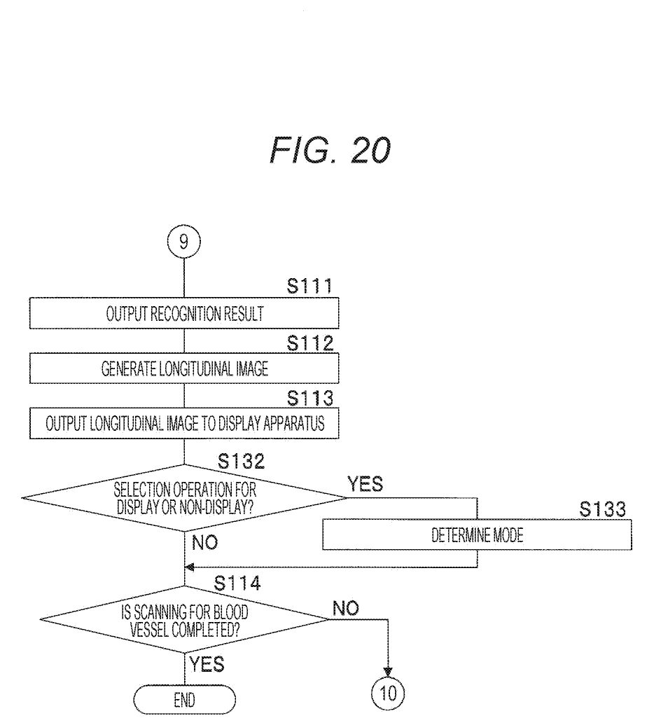

FIGS. 18 to 20 are flowcharts illustrating examples of the processes executed by the image processing apparatus 3 according to the third embodiment. When an operation to start scanning by the image diagnosis apparatus 100 is performed via the input apparatus 5 and a signal is output from the imaging device 11 of the catheter 1, the processing unit 30 of the image processing apparatus 3 starts the following processing. Among the steps illustrated in the flowcharts of FIGS. 18 to 20, steps common to the process illustrated in the flowcharts of FIGS. 5 and 6 of the first embodiment are denoted by the same step numbers, and detailed description thereof will be omitted.

The processing unit 30 of the image processing apparatus 3 stores a signal data, the tomographic image I1 generated based on the signal data, and the recognition result based on the segmentation model 31M in the storage unit 31 (S105). Before generating the tomographic images of different display depths, the processing unit 30 determines whether the mode is: the first mode in which both the tomographic image I11 of the first display depth and the tomographic image I12 of the second display depth are displayed, the second mode in which only the tomographic image I11 of the first display depth is displayed, or the third mode in which only the tomographic image I12 of the second display depth is displayed (S131). Initially, the first mode is stored in the storage unit 31 as setting data.

In a case where the mode is determined to be the first mode (S131: first mode), the processing unit 30 generates the tomographic image I11 of the first display depth and the tomographic image I12 of the second display depth, which are different in the radial direction, from the tomographic image I1 (S106).

The processing unit 30 executes image processing on the generated tomographic image I11 of the first display depth based on the image setting corresponding to the first display depth (S107), and executes image processing on the tomographic image I12 of the second display depth based on the image setting corresponding to the second display depth (S108).

The processing unit 30 outputs the tomographic image I11 of the first display depth subjected to the image processing and the tomographic image I12 of the second display depth subjected to the image processing such that they can be displayed in real time on the screen displayed on the display apparatus 4 (S109). The processing unit 30 stores the tomographic image I11 of the first display depth subjected to the image processing and the tomographic image I12 of the second display depth subjected to the image processing (S110).

The processing unit 30 outputs text indicating the recognition result to the display apparatus 4 (S111). The processing unit 30 generates the longitudinal image I2 (S112) and outputs the generated longitudinal image I2 to the display apparatus 4 (S113).

The processing unit 30 determines whether a display/non-display selecting operation is received in a state where one of the tomographic images I11 and I12 is being displayed on the display apparatus 4 (S132). The processing unit 30 may receive the display/non-display selection operation using display/non-display buttons respectively corresponding to the first display depth and the second display depth, the display/non-display buttons being provided in the input apparatus 5. The processing unit 30 may display a cursor on the screen displayed on the display apparatus 4, and may receive a selection of display/non-display in a menu displayed when, for example, an operation corresponding to a right click of a mouse is performed on one of the tomographic images I11 and I12 with the cursor.

In a case where it is determined in S132 that the display/non-display selection operation has not been received (S132: NO), the processing unit 30 advances the processing to S114.

In a case where it is determined in S132 that the display/non-display selection operation has been received (S132: YES), the processing unit 30 determines the mode to be one of the first mode, the second mode, and the third mode in accordance with the target for which display is selected (S133). In S133, in a case where both the tomographic image I11 of the first display depth and the tomographic image I12 of the second display depth are initially displayed in the first mode, when the non-display selection operation is performed for one of the tomographic images I11 and I12, the processing unit 30 determines the mode to be the second mode or the third mode in accordance with the target. In the second mode in which only the tomographic image I11 of the first display depth is displayed, the non-display of the tomographic image I11 of the first display depth as the target is disabled. Similarly, in the third mode in which only the tomographic image I12 of the second display depth is displayed, the non-display of the tomographic image I12 of the second display depth as the target is disabled. After the determination in S133, the processing unit 30 advances the processing to S114.

In a case where it is determined in S131 that the mode is the second mode (S131: second mode), the processing unit 30 generates the tomographic image I11 of the first display depth for mainly observing the blood vessel from the tomographic image I1 (S134). The processing unit 30 executes image processing on the generated tomographic image I11 of the first display depth based on the image setting corresponding to the first display depth (S135).

The processing unit 30 outputs the tomographic image I11 of the first display depth after the execution of the image processing in S135 such that the image can be displayed in real time on the screen displayed on the display apparatus 4 (S136). The processing unit 30 stores the tomographic image of the first display depth subjected to the image processing in the storage unit 31 in association with the data of the position on the longitudinal axis in the blood vessel (S137). Since the tomographic image I1 has already been stored in the storage unit 31, the processing of S137 is not essential. The processing unit 30 advances the processing to S111.

In a case where it is determined in S131 that the mode is the third mode (S131: third mode), the processing unit 30 generates the tomographic image I12 of the second display depth for mainly observing the outside of the blood vessel from the tomographic image I1 (S138). The processing unit 30 executes image processing on the generated tomographic image I12 of the second display depth based on the image setting corresponding to the second display depth (S139).

The processing unit 30 outputs the tomographic image I12 of the second display depth after the execution of the image processing in S139 such that the image can be displayed in real time on the screen displayed on the display apparatus 4 (S140). The processing unit 30 stores the tomographic image of the second display depth subjected to the image processing in the storage unit 31 in association with the data of the position on the longitudinal axis in the blood vessel (S141). Since the tomographic image I1 has already been stored in the storage unit 31, the processing of S141 is not essential. The processing unit 30 advances the processing to S111.

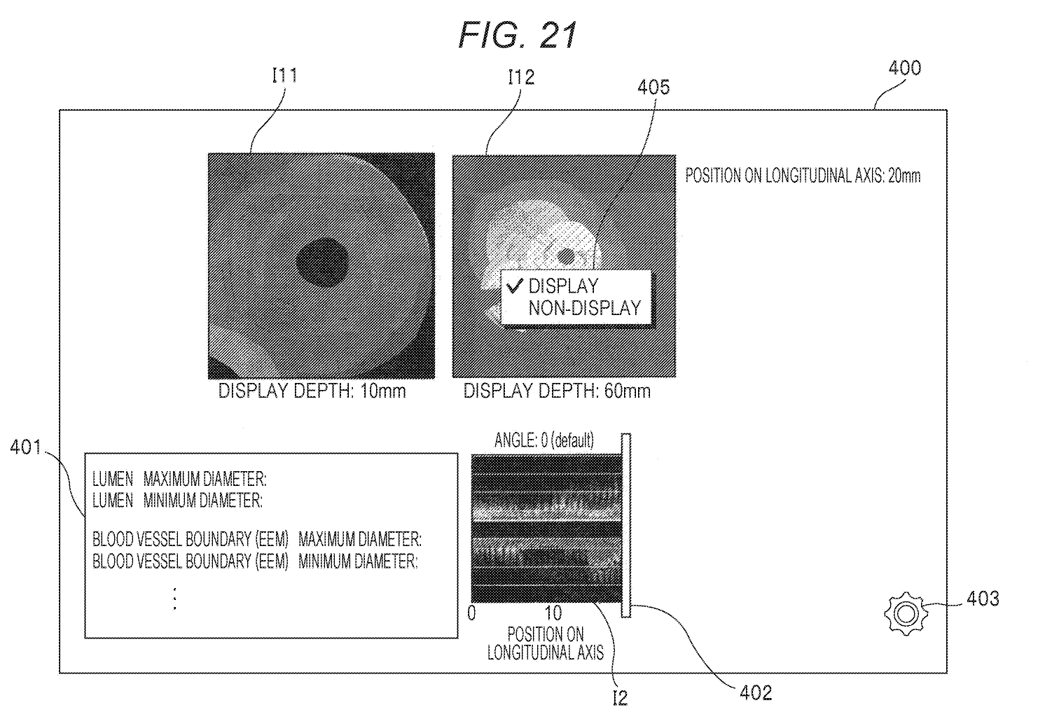

FIG. 21 is a diagram illustrating an example of the screen 400 displayed on the display apparatus 4 in the third embodiment. Similar to the example of the screen 400 illustrated in FIG. 11 of the first embodiment, the screen 400 illustrated in FIG. 21 includes at least the tomographic image I11 and includes the text 401 indicating data of anatomical features, the longitudinal image I2, and the cursor 402.

The screen 400 illustrated in FIG. 21 includes a menu 405 which is displayed when a cursor is superimposed on the tomographic image I12 among the tomographic images I11 and I12 of different display depths on the screen displayed on the display apparatus 4 and a specific operation (right click of the mouse) is performed. In the menu 405, “display” is selected on the screen 400 illustrated in FIG. 21, but when the examination operator or the physician selects “non-display” using the input apparatus 5, the screen 400 changes to a screen on which only the tomographic image I11 of the first display depth is displayed as illustrated in FIG. 16 of the second embodiment.

The display/non-display of the tomographic image I11 of the first display depth and the tomographic image I12 of the second display depth may be selected by speech recognition using an audio input/output unit included in the input apparatus 5. Thus, while performing the operation to drive the catheter 1, the examination operator or the physician can visually recognize the tomographic images I11 and I12 subjected to the appropriate image processing using the display apparatus 4 and switch the display details in accordance with the position and the observation details.

### Modification Example

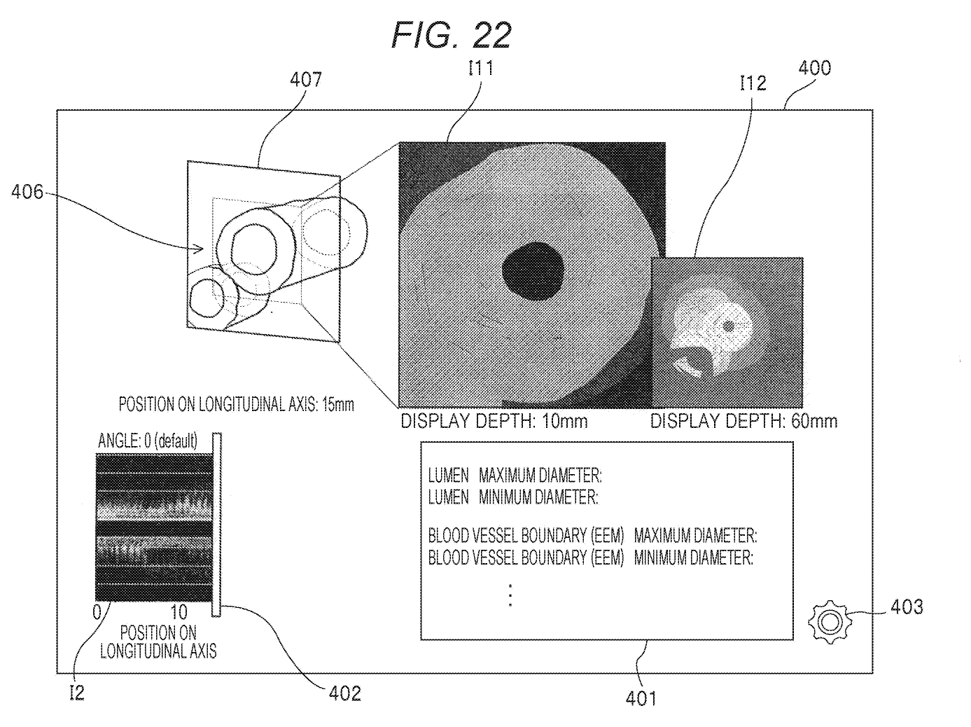

In the first to third embodiments, examples of processing have been described by exemplifying the screen 400 including the tomographic images I11 and I12, and the longitudinal image I2. The display of the tomographic images I11 and I12, and the longitudinal image I2 may be combined with a three-dimensional image. FIG. 22 is a diagram illustrating an example of the screen 400 according to a modification example. In the modification example, the screen 400 includes a three-dimensional image 406 generated based on the recognition result, and includes a three-dimensional cursor 407 indicating a position on a longitudinal axis in the three-dimensional image 406. The three-dimensional image 406 in the screen 400 of FIG. 22 may be constantly output as illustrated in the first embodiment, or may be output together with the tomographic image I12 only in a case where it is determined that another organ (artery) exists outside the blood vessel and within the range of the second display depth as illustrated in the second embodiment.

As described above, in a case where it is necessary to identify the structure of a target organ relative to surrounding organs, such as in a case where a vein is an observation target, the tomographic image I11 of the first display depth and the tomographic image I12 of the second display depth may be generated and displayed in a timely manner. As in the modification example, by outputting a three-dimensional structure using the three-dimensional image, the organ to be observed can be more appropriately understood. Moreover, by performing appropriate image processing on both the inside and the outside of the blood vessel depending on the difference in display depth, it is possible to appropriately display information regarding the interior and the exterior of the blood vessel.

The embodiments disclosed as above are illustrative in all respects and are not restrictive. The scope of the present disclosure is defined by the claims, and is intended to include all modifications within the meaning and scope equivalent to the claims.

What is claimed is:

1. An image diagnosis apparatus comprising:
a connection interface connectable to an image diagnosis catheter for a luminal organ, the image diagnosis catheter including an ultrasound transmitter and receiver;
a display;
a memory that stores a program and setting data, the setting data indicating a first display depth, a first image setting corresponding to the first display depth, a second display depth different from the first display depth, and a second image setting corresponding to the second display depth; and
a processor configured to execute the program to perform the steps of:
receiving a signal acquired from the ultrasound transmitter and receiver via the connection interface,
converting a distribution of reflected waves in a radial direction indicated by the received signal into a brightness distribution, and generating a base tomographic image of the luminal organ based on the brightness distribution,
generating, from the base tomographic image, first and second tomographic images of the luminal organ at the first and second display depths, respectively,
executing image processing on the first and second tomographic images using the first and second image settings, respectively,
generating a screen showing the processed first and second tomographic images along with information indicating a position of the luminal organ corresponding to the processed first and second tomographic images, and
controlling the display to display the generated screen.

2. The image diagnosis apparatus according to claim 1, wherein
the first display depth corresponds to a range encompassing an interior of the luminal organ, and
the second display depth corresponds to a range extending outside the luminal organ to encompass another organ existing outside the luminal organ.

3. The image diagnosis apparatus according to claim 1, wherein
each of the first and second image settings includes at least one of a gain setting, a contrast setting, a sensitivity time control (STC) setting, and a gamma correction setting, and
executing the image processing includes adjusting the first and second tomographic images based on the at least one of the gain setting, the contrast setting, the STC setting, and the gamma correction setting in the respective first and second image settings.

4. The image diagnosis apparatus according to claim 1, wherein
the steps include generating a longitudinal tomographic image in an axial direction of the luminal organ based on the received signal, and
the screen further shows the longitudinal tomographic image.

5. The image diagnosis apparatus according to claim 4,
wherein the information indicating the position of the luminal organ includes a cursor superimposed on the longitudinal tomographic image to indicate the position of the luminal organ corresponding to the processed first and second tomographic images.

6. The image diagnosis apparatus according to claim 1,
wherein the steps include receiving a user input for setting the first and second display depths.

7. The image diagnosis apparatus according to claim 1, wherein
the second display depth is greater than the first display depth,
the steps include determining, for different positions in an axial direction of the luminal organ, whether another organ exists within a distance corresponding to the second display depth from the luminal organ, and
the step of generating the screen showing the processed first and second tomographic images is performed only for a position where it is determined that said another organ exists.

8. The image diagnosis apparatus according to claim 1, wherein the steps include:
receiving a selection operation for displaying or not displaying each of the processed first and second tomographic images, and
updating the screen to hide the at least one of the processed first and second tomographic images in accordance with the received operation.

9. The image diagnosis apparatus according to claim 1, wherein the steps include dynamically switching between displaying only the processed first tomographic image and displaying both the processed first and second tomographic images on the generated screen in response to a change in position of the image diagnosis catheter during movement in an axial direction of the luminal organ.

10. The image diagnosis apparatus according to claim 1,
wherein
the memory further stores a trained segmentation model,
the steps include:
inputting the base tomographic image into the trained segmentation model to identify a region corresponding to the luminal organ, and
generating text data indicating anatomical features of the luminal organ based on an output from the trained segmentation model, and
the generated screen further shows the generated text data.

11. An image diagnosis method comprising:
storing, in a memory, setting data indicating a first display depth, a first image setting corresponding to the first display depth, a second display depth different from the first display depth, and a second image setting corresponding to the second display depth;
receiving a signal acquired from an image diagnosis catheter having an ultrasound transmitter and receiver and inserted into a luminal organ;
converting a distribution of reflected waves in a radial direction indicated by the received signal into a brightness distribution, and generating a base tomographic image of the luminal organ based on the brightness distribution;
generating, from the base tomographic image, first and second tomographic images of the luminal organ at the first and second display depths, respectively;
executing image processing on the first and second tomographic images using the first and second image settings, respectively; and
displaying a screen showing the processed first and second tomographic images along with information indicating a position of the luminal organ corresponding to the processed first and second tomographic images.

12. The image diagnosis method according to claim 11, wherein
the first display depth corresponds to a range encompassing an interior of the luminal organ, and
the second display depth corresponds to a range extending outside the luminal organ to encompass another organ existing outside the luminal organ.

13. The image diagnosis method according to claim 11, wherein
each of the first and second image settings includes at least one of a gain setting, a contrast setting, a sensitivity time control (STC) setting, and a gamma correction setting, and
executing the image processing includes adjusting the first and second tomographic images based on the at least one of the gain setting, the contrast setting, the STC setting, and the gamma correction setting in the respective first and second image settings.

14. The image diagnosis method according to claim 11, further comprising:
generating a longitudinal tomographic image in an axial direction of the luminal organ based on the received signal, wherein
the screen further shows the longitudinal tomographic image.

15. The image diagnosis method according to claim 14, wherein the information indicating the position of the luminal organ includes a cursor superimposed on the longitudinal tomographic image to indicate the position of the luminal organ corresponding to the processed first and second tomographic images.

16. The image diagnosis method according to claim 11, further comprising:
receiving a user input for setting the first and second display depths.

17. The image diagnosis method according to claim 11, wherein
the second display depth is greater than the first display depth, and
the image diagnosis method further comprises:
determining, for different positions in an axial direction of the luminal organ, whether another organ exists within a distance corresponding to the second display depth from the luminal organ, and
generating the screen showing the processed first and second tomographic images is performed only for a position where it is determined that said another organ exists.

18. The image diagnosis method according to claim 11, further comprising:
receiving a selection operation for displaying or not displaying each of the processed first and second tomographic images; and
updating the screen to hide the at least one of the processed first and second tomographic images in accordance with the received operation.

19. The image diagnosis method according to claim 11, further comprising:
dynamically switching between displaying only the processed first tomographic image and displaying both the processed first and second tomographic images on the generated screen in response to a change in position of the image diagnosis catheter during movement in an axial direction of the luminal organ.

20. A non-transitory computer-readable storage medium storing a program that causes a processor to execute a process comprising:
storing, in a memory, setting data indicating a first display depth, a first image setting corresponding to the first display depth, a second display depth different from the first display depth, and a second image setting corresponding to the second display depth;
receiving a signal acquired from an image diagnosis catheter having an ultrasound transmitter and receiver and inserted into a luminal organ;
converting a distribution of reflected waves in a radial direction indicated by the received signal into a brightness distribution, and generating a base tomographic image of the luminal organ based on the brightness distribution;
generating, from the base tomographic image, first and second tomographic images of the luminal organ at the first and second display depths, respectively;
executing image processing on the first and second tomographic images using the first and second image settings, respectively; and
displaying a screen showing the processed first and second tomographic images along with information indicating a position of the luminal organ corresponding to the processed first and second tomographic images.
